(19) United States
(12) Patent Application Publication
(10) Pub. No.: US 2007/0078463 A1
(43) Pub. Date: Apr. 5, 2007

(54) Retractable cannula and method for minimally invasive medical procedure

(76) Inventor: Hugues F. Malandain, Mountain View, CA (US)

Correspondence Address: COOLEY GODWARD KRONISH LLP;ATTN: PATENT GROUP, Suite 500, 1200 - 19th Street, NW, WASHINGTON, DC, 20036-2402, US

(21) Appl. No.: 11/384,514

(22) Filed: Mar. 21, 2006

Related U.S. Application Data
(60) Provisional application No. 60/696,805, filed on Jul. 7, 2005.

(51) Int. Cl. A61B 17/00

(52) U.S. Cl. 606/80

(57) ABSTRACT

Apparatuses and methods for performing minimally invasive medical procedures are disclosed herein. In one example, an apparatus includes an outer elongate body including a lumen extending from a proximal portion to a distal portion of the outer elongate body. An inner elongate body is slidably received within the lumen of the outer elongate body. A distal portion of the inner elongate body is configured to be inserted into a tissue. The outer elongate body and the inner elongate body collectively define a channel to removably receive a tool when the outer elongate body and the inner elongate body are slidably extended from each other. At least one of the tool and the inner elongate body are configured to puncture the tissue.

### CROSS-REFERENCE TO RELATED APPLICATION

This application claims priority to U.S. Provisional Application Ser. No. 60/696,805 entitled “Retractable Cannula and Method for Minimally Invasive Medical Procedure,” filed Jul. 7, 2005, the disclosure of which is incorporated herein by reference in its entirety.

### BACKGROUND

The invention relates generally to medical devices and procedures, and more particularly to a minimally invasive access cannula and method for accessing a body of a patient to perform a medical procedure.

Various known medical devices are configured to provide access to a specific bone or tissue site within a body of a patient. Known devices, such as cannulas, typically include at least one channel for inserting a medical tool to perform a minimally-invasive (e.g., percutaneous) medical procedure. For example, in a medical procedure performed on a vertebra, access to the vertebra is typically achieved by penetrating percutaneously the vertebra via a stylet or other medical tool having a sharp tip. A cannula is then inserted into the vertebra to provide a working channel for the physician to access the vertebra. Access cannulas are typically available in one or two sizes. Depending on the size of the patient, the cannula may be too long and protrude outside of the patient's body by a few inches. This creates an obstacle in the working area for the physician. If the physician hits or bumps the portion of the cannula protruding outside of the body of the patient, the sudden movement of the cannula can create a moment at the base of the insertion into the bone, resulting in inadvertent damage to the vertebra.

Thus, a need exists for an adjustable access cannula for use in minimally-invasive medical procedures that eliminates the above described problems. An adjustable cannula that can be lengthened or shortened allows a physician to adjust the length of the cannula as needed for the particular patient.

### SUMMARY OF THE INVENTION

Apparatuses and methods for performing minimally invasive medical procedures are disclosed herein. In one example, an apparatus includes an outer elongate body including a lumen extending from a proximal portion to a distal portion of the outer elongate body. An inner elongate body is slidably received within the lumen of the outer elongate body. A distal portion of the inner elongate body is configured to be inserted into a tissue. The outer elongate body and the inner elongate body collectively define a channel to removably receive a tool when the outer elongate body and the inner elongate body are slidably extended from each other. At least one of the tool and the inner elongate body are configured to puncture the tissue.

### BRIEF DESCRIPTION OF THE DRAWINGS

The present invention is described with reference to the accompanying drawings. In the drawings, like reference numbers indicate identical or functionally similar components.

FIG. 1 is a schematic illustrating a medical device according to an embodiment of the invention.

FIGS. 2 and 3 are cross-sectional views of a medical device according to an embodiment of the invention shown in a telescopically extended configuration and a telescopically collapsed configuration, respectively.

FIG. 4 is a cross-sectional view of a medical device according to another embodiment of the invention.

FIG. 5 is a perspective view of a portion of an inner elongate body according to an embodiment of the invention.

FIG. 6 is a cut-away perspective view of a portion of an outer elongate body according to an embodiment of the invention.

FIG. 7 is a cross-sectional view of a medical device according to another embodiment of the invention shown in an extended configuration.

FIG. 8 is a cross-sectional view of a portion of a medical device according to another embodiment of the invention.

FIG. 9 is a cross-sectional view of a medical device according to yet another embodiment of the invention.

FIG. 10 is a cross-sectional view of the medical device shown in FIG. 9 taken along line 10-10 in FIG. 9.

FIG. 11 is a cross-sectional view of a medical device according to another embodiment of the invention.

FIG. 12 is a cross-sectional view of the medical device shown in FIG. 11 taken along line 12-12 in FIG. 11.

FIG. 13 is a cross-sectional view of a portion of a medical device according to another embodiment of the invention, illustrating a collar in a closed configuration.

FIG. 14 is a cross-sectional view of the portion of a medical device shown in FIG. 11 illustrating the collar in an open configuration.

FIG. 15 is side view of a medical device according to an embodiment of the invention.

FIG. 16 is a cross-sectional side view of a portion of the medical device shown in FIG. 7 in a partially collapsed configuration.

### DETAILED DESCRIPTION

An apparatus (also referred to herein as a “medical device”) can be used in a variety of minimally-invasive (e.g., percutaneous, mini-open, endoscopic) medical procedures, such as a vertabroplasty or Kyphoplasty procedure in a vertebra-related procedure. The medical device may also be used for medical procedures performed in other areas of a patient. The following description focuses on use of the medical device in a vertebra procedure, but it should be understood that procedures on other areas of a body, including other hard tissue (e.g., bone structures) and soft tissue areas, may be performed with the medical device.

As used in this specification and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the cannula end inserted inside the patient's body would be the distal end of the cannula, while the cannula end outside the patient's body would be the proximal end of the cannula.

In one embodiment, where the medical device is configured to be inserted percutaneously into a tissue (e.g., a vertebral body), the medical device provides access to the tissue as part of a medical procedure. The medical device includes a channel for inserting a medical tool to perform the medical procedure. The medical device includes an inner elongate body and an outer elongate body that are slidably (e.g., telescopically) coupled to provide length adjustability of the medical device. The inner elongate body is configured to be inserted into a portion of a tissue. The outer elongate body is configured to collapse over the inner elongate body to minimize the portion of the medical device protruding outside of the body of the patient. The adjustability of the medical device provides for an improved work area for the physician and reduces the risks of damaging the tissue during the medical procedure.

In another embodiment, the medical device includes an inner elongate body and an outer elongate body that are slidably coupled to provide length adjustability of the medical device. In this embodiment, the inner elongate body and the outer elongate body are configured to lock in a fully extended configuration such that the outer elongate body and the inner elongate body are collectively rotatable without rotating with respect to each other. In the extended configuration, the medical device can be inserted into the tissue with better control, since the inner elongate body and the outer elongate body are unable to rotate relative to each other. This configuration may be optional for medical procedures involving soft tissue, where the medical device can be inserted into the tissue with relative ease.

In another embodiment, the medical device includes an outer elongate body, an inner elongate body and a tool, such as a stylet. The inner elongate body and the outer elongate body are slidably (e.g., telescopically) coupled and collectively define a channel when in a slidably extended configuration. The channel has a length when the inner elongate body and the outer elongate body are in the extended configuration. The tool is configured to be received within the channel and has a length greater than the length of the channel while the outer elongate body and the inner elongate body are in the extended configuration.

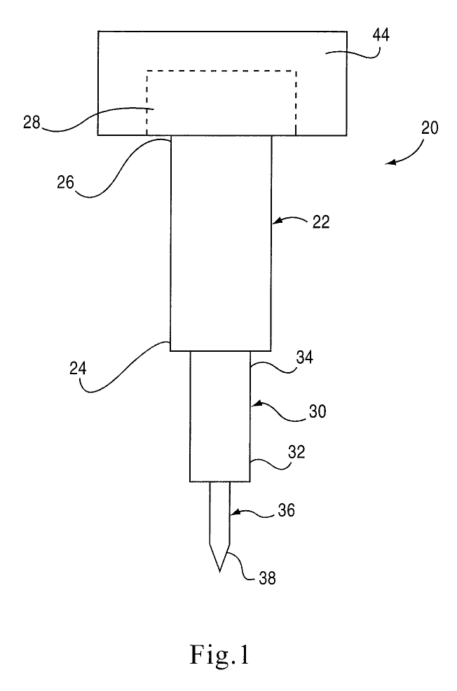

FIG. 1 is a schematic illustration of a medical device 20 according to an embodiment of the invention. The medical device 20 includes an outer elongate body 22 and an inner elongate body 30. The outer elongate body 22 includes a proximal end portion 26 and a distal end portion 24, and defines a lumen (not shown in FIG. 1) extending from the proximal end portion 26 to the distal end portion 24. The inner elongate body 30 includes a proximal end portion 34 and a distal end portion 32, and defines a lumen (not shown in FIG. 1) extending from the proximal end portion 34 to the distal end portion 32. The distal end portion 32 of the inner elongate body 30 is configured to be inserted into a portion of a tissue, such as a vertebra.

The inner elongate body 30 is configured to be slidably (e.g., telescopically) received within the lumen of the outer elongate body 22. The inner elongate body 30 and the outer elongate body 22 collectively have an extended configuration, and an extraction or collapsed configuration. In the extended configuration, the outer elongate body 22 and the inner elongate body 30 are fully extended with respect to each other and have a maximum collective length. In the extraction configuration, the outer elongate body 22 and the inner elongate body 30 are at least partially slidably collapsed with respect to each other. When in the extended configuration, the inner elongate body 30 is configured to couple to the outer elongate body 22 such that the outer elongate body 22 and the inner elongate body 30 are collectively rotatable without the outer elongate body and the inner elongate body 30 rotating with respect to each other.

The medical device 20 may also include a tool 36 that is configured to be removably received within a channel (not shown in FIG. 1) defined collectively by the inner elongate body 30 and the outer elongate body 22. The tool 36 can be, for example, a stylet or other piercing tool. The tool 36 has a length that is greater than a length of the channel defined collectively by the inner elongate body 30 and the outer elongate body 22 while in the extended configuration. The tool 36 may include a handle 44 configured to releasably and matingly couple to a handle 28 of the outer elongate body 22. The tool 36 can include a sharp tip 38 configured to puncture the tissue when the inner elongate body 30 and the outer elongate body 22 are slidably extended from each other in the extended configuration. In an alternative embodiment, the inner elongate body includes a sharp tip configured to puncture the tissue instead of the tool, as will be described in an alternative embodiment below. In some embodiments, the inner elongate body 30 can include a handle (not shown in FIG. 1) that can releasably and matingly couple to the outer elongate body 22.

In use, the medical device 20 is percutaneously inserted into a body of a patient while the inner elongate body 30 and the outer elongate body 22 are in the extraction configuration and the handle 44 of the tool 36 is coupled to the handle 28 of the outer elongate body 22. In the embodiment shown in FIG. 1, the sharp tip 28 of the tool 36 punctures a portion of the tissue to create a path for the distal end portion 32 of the inner elongate body 30 to be inserted into a portion of the tissue. When a portion of the medical device 20 is positioned within the tissue, the tool 36 is removed from the channel by uncoupling the handle 44 of the tool 36 from the handle 28 of the outer elongate body 22, and pulling the tool 36 out proximally. The outer elongate body 22 can then be slidably collapsed over the inner elongate body 30 to adjust the length of the medical device 20. The adjustability of the medical device 20 allows the handle 28 of the outer elongate body 22 to be positioned at a selected height above the body surface of the patient. Thus, the handle 28 of the outer elongate body 22 may be positioned substantially close to the body surface of the patient, eliminating the problems described above when an access cannula protrudes into the working area of the physician.

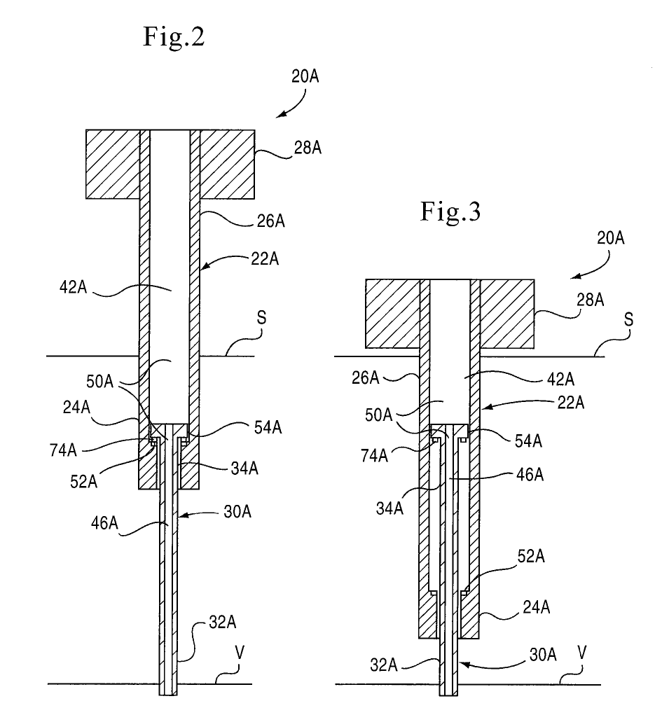

FIGS. 2 and 3 illustrate cross-sectional views of a medical device 20A according to an embodiment of the invention in an extended configuration and in a collapsed or extraction configuration, respectively. The medical device 20A includes an outer elongate body 22A and an inner elongate body 30A. In this embodiment, the outer elongate body 22A includes a proximal end portion 26A, a distal end portion 24A and defines a lumen 42A. The outer elongate body 22A also includes a handle 28A. The inner elongate body 30A includes a proximal end portion 34A, a distal end portion 32A and defines a lumen 46A.

The medical device 20A is shown inserted through the outer surface S of a patient such that the distal end portion 32A of the inner elongate body 30A is inserted into a portion of a vertebra V. In this embodiment, the proximal end portion 34A of the inner elongate body 30A defines a flange 54A. The distal end portion 24A of the outer elongate body 22A defines a flange 52A. The flange 54A on the proximal end portion 34A of the inner elongate body 30A matingly couples to the flange 52A on the distal end portion 24A of the outer elongate body 22A. A release mechanism 74A releasably holds the inner elongate body 30A and the outer elongate body 22A in the extended configuration, as shown in FIG. 2. Release mechanism 74A may include mating components on the inner elongate body 30A and the outer elongate body 22A, such as friction fit components, snap fit connectors, mating keyway and key, or any other known releasable coupling.

FIG. 3 illustrates the medical device 20A in the collapsed configuration in which the outer elongate body 22A is collapsed over the inner elongate body 30A and in which the handle 28A of the outer elongate body 22A is positioned at a selected height above the body surface S. From this position, the physician can insert a medical tool through the lumen 42A of the outer elongate body 22A and the lumen 46A of the inner elongate body 30A (collectively defining a channel 50A) to perform a medical procedure on at least a portion of the vertebra V. In an alternative arrangement, the handle 28A of the outer elongate body 22A is positioned flush with the body surface S.

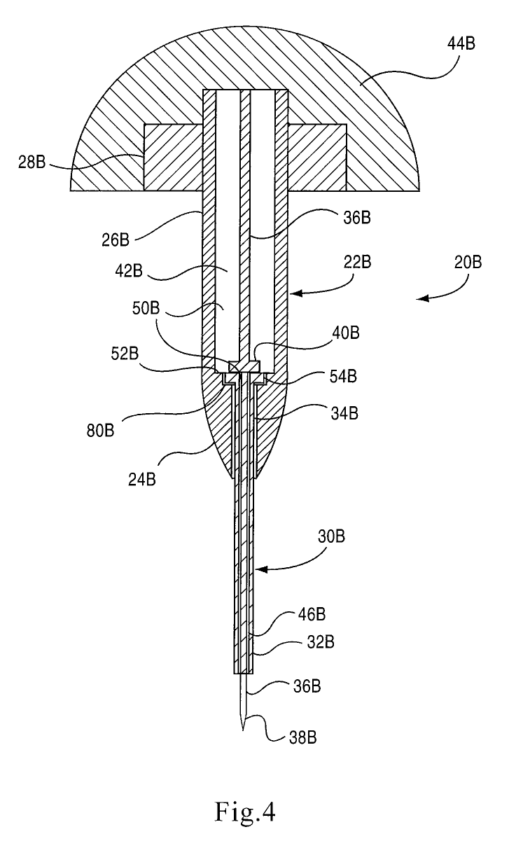

FIG. 4 illustrates a medical device 20B according to another embodiment of the invention. Medical device 20B has a substantially similar structure and performs a substantially similar function as the previous embodiments. Medical device 20B includes an outer elongate body 22B, an inner elongate body 30B, and a tool 36B. The outer elongate body 22B includes a proximal end portion 26B and a distal end portion 24B, and defines a lumen 42B extending from the proximal end portion 26B to the distal end portion 24B. The inner elongate body 30B includes a proximal end portion 34B and a distal end portion 32B, and defines a lumen 46B extending from the proximal end portion 34B to the distal end portion 32B.

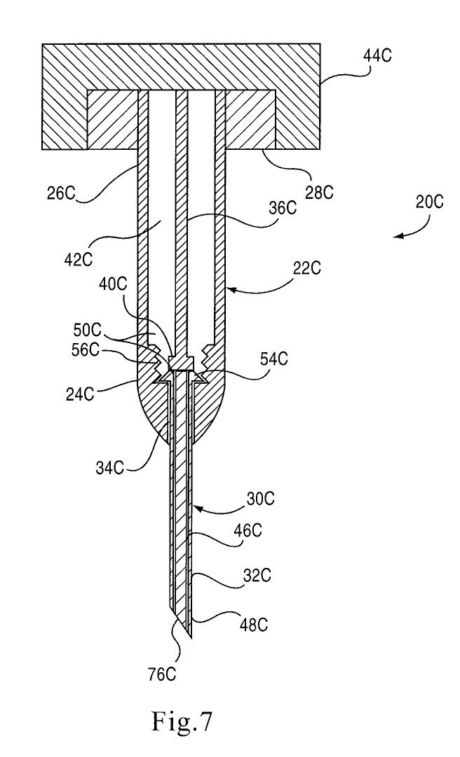

The tool 36B is configured to be received within the lumen 42B of the outer elongate body 22B and the lumen 46B of the inner elongate body 30B (collectively defining a channel 50B). The tool 36B extends distally from the inner elongate body 30B and in this embodiment includes a sharp tip 38B configured to puncture a tissue such as, for example, a vertebra. In alternative embodiments, the tool 36B does not have a sharp tip and is used as a block to prevent material from the tissue from entering the lumen 46B when the inner elongate body 30B is inserted into the tissue (see also FIG. 7). The tool 36B also includes a handle 44B configured to matingly and releasably couple to a handle 28B of the outer elongate body 22B. The tool 36B includes a shoulder 40B configured to contact a flange 54B on a proximal end portion 34B of the inner elongate body 30B. The shoulder 40B contacts the inner elongate body 30B while having the inner elongate body 30B distally within the lumen 42B of the outer elongate body 22B, and reduces or prevents forces from being transferred to the outer elongate body 22B when the medical device 20B impacts a tissue. In this embodiment, the shoulder 40B also allows the distal movement of the inner elongate body 30B without requiring a locking relationship between tool 36B and inner elongate body 30B

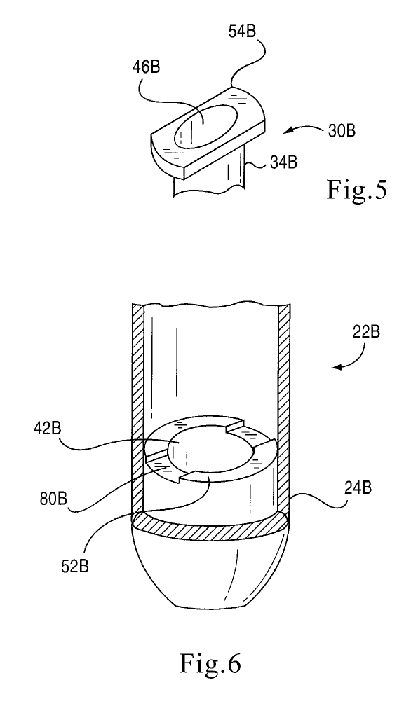

In the embodiment shown in FIG. 4, the flange 54B on the inner elongate body 30B is configured to matingly couple to a cutout 80B on a flange 52B on the distal end portion 24B of the outer elongate body 22B. FIG. 5 illustrates a portion of the proximal end portion 34B of the inner elongate body 30B, and FIG. 6 illustrates a cut-out section of the distal end portion 24B of the outer elongate body 22B. When the flange 54B is matingly positioned within the cutouts 80B, the inner elongate body 30B and the outer elongate body 22B are prevented from rotating relative to each other. When the tool 36B is positioned within the channel 50B and the handle 44B of the tool 36B is coupled to the handle 28B of the outer elongate body 22B, the medical device 20B will be in an extended configuration. In this configuration, the shoulder 40B on the tool 36B prevents the inner elongate body 30B from moving axially within the lumen 42B of the outer elongate body 22B. In addition, in the extended configuration the inner elongate body 30B and the outer elongate body 22B can collectively rotate. When the tool 36B is removed, the flange 54B of the inner elongate body 30B can be uncoupled from the cutouts 80B on the outer elongate body 22B. The outer elongate body 22B can then be collapsed over the inner elongate body 30B to adjust the length of the medical device 20B.

FIG. 7 illustrates a cross-sectional view of a medical device 20C according to another embodiment of the invention. Medical device 20C is a similar structure and performs a similar function as the previous embodiments. Medical device 20C includes an outer elongate body 22C having a proximal end portion 26C, a distal end portion 24C, and defines a lumen 42C extending from the proximal end portion 26C to the distal end portion 24C. The distal end portion 24C includes a threaded portion 56C on an interior wall. The proximal end portion 26C of the outer elongate body 22C includes a handle 28C. An inner elongate body 30C includes a proximal end portion 34C and a distal end portion 32C, and defines a lumen 46C extending from the proximal end portion 34C to the distal end portion 32C. The inner elongate body 30C defines an opening 76C on the distal end portion 32C that communicates with lumen 46C. The distal end portion 32C includes a sharp tip portion 48C configured to puncture a tissue, such as, for example a vertebra.

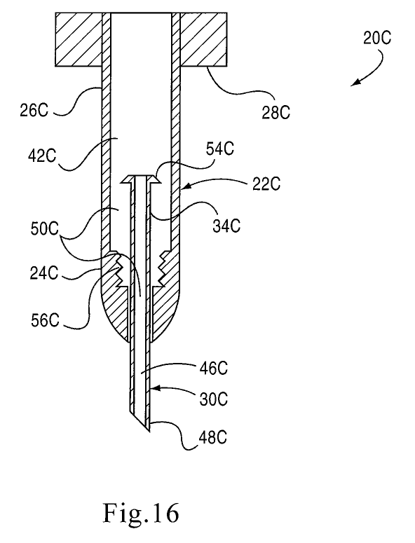

In this embodiment, a flange 54C on the proximal end portion 34C of the inner elongate body 30C is configured to threadedly couple to the threaded portion 56C of the outer elongate body 22C. The threaded coupling of the outer elongate body 22C and the inner elongate body 30C locks the medical device 20C in an extended configuration as previously described such that the outer elongate body 22C and the inner elongate body 30C can collectively rotate without being able to rotate relative to each other. To uncouple the threaded coupling of the inner elongate body 30C and the outer elongate body 22C, the outer elongate body 22C is turned to disengage the proximal end portion 34C of the inner elongate body 30C from the threaded portion 56C of the outer elongate body 22C, as illustrated in FIG. 16. The outer elongate body 22C can then be slidably (e.g., telescopically) collapsed over the inner elongate body 30C to adjust the length of the medical device 20C, and position the handle 28C at a selected height above the body surface of the patient.

A tool 36C may optionally be included. The tool 36C is configured to be received within the lumen 42C of the outer elongate body 22C and the lumen 46C of the inner elongate body (collectively defining a channel 50C). The tool 36C extends distally to the opening 76C defined by the inner elongate body 30C. The tool 36C serves as a block within the inner elongate body 30C to prevent material from the vertebra from entering lumen 46C when the inner elongate body 30C is puncturing the tissue. A shoulder 40C on tool 36C is configured to contact flange 54C on the inner elongate body 30C and limits how far distally the tool 36C can extend within the inner elongate body 30C. The tool 36C also includes a handle 44C configured to matingly and releasably couple to handle 28C on the outer elongate body 22C.

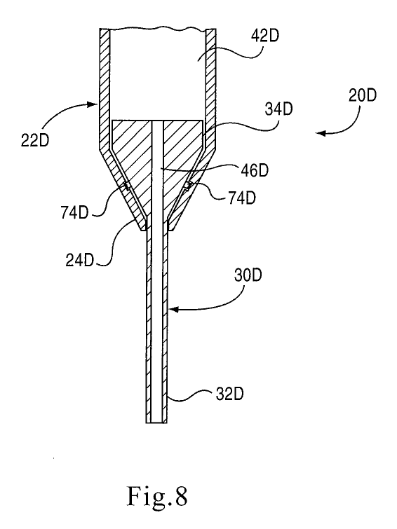

FIG. 8 illustrates a portion of a medical device 20D according to yet another embodiment of the invention. The medical device 20D includes an outer elongate body 22D and an inner elongate body 30D. The outer elongate body 22D) includes a distal end portion 24D and a proximal end portion (not shown in FIG. 8), and defines a lumen 42D. The inner elongate body 30D includes a proximal end portion 34D and a distal end portion 32D, and defines a lumen 46D. The medical device 20D is similar to medical device 20A except in this embodiment the proximal end portion 34D of the inner elongate body 30D includes a tapered shape that couples with a mating tapered shape on the distal end portion 24D of the outer elongate body 22D. A release mechanism 74D may be included to lock the medical device 20D in the extended configuration as described for medical device 20A. Medical device 20D can optionally include a tool, such as a stylet (not shown) (e.g., similar to tool 36B or tool 36C) that can be removably inserted into lumen 42D of outer elongate body 22D and lumen 46D of inner elongate body 30D. Similar to the previous embodiments, the tool can have a shoulder (e.g., shoulder 40B or shoulder 40C) configured to contact the distal end portion 34D of inner elongate body 30D.

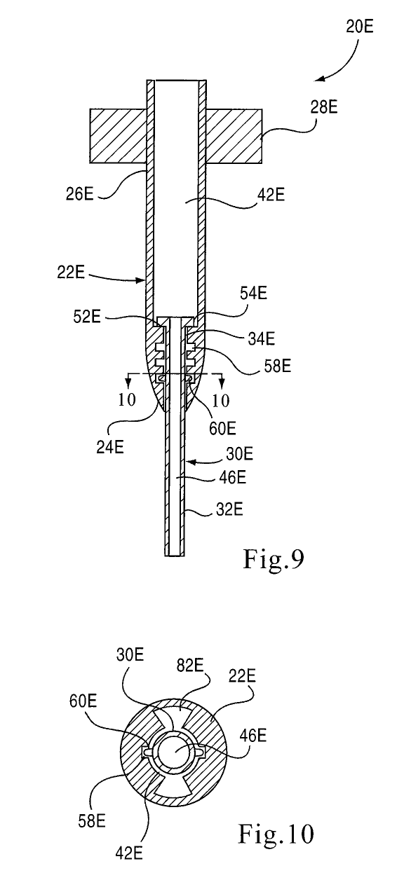

FIGS. 9 and 10 illustrate cross-sectional views of a medical device 20E according to still another embodiment of the invention. In this embodiment, the medical device 20E includes an outer elongate body 22E and an inner elongate body 30E. The outer elongate body 22E includes a proximal end portion 26E and a distal end portion 24E, and defines a lumen 42E. The inner elongate body 30E includes a proximal end portion 34E and a distal end portion 32E, and defines a lumen 46E. The distal end portion 24E on the outer elongate body 22E includes multiple apertures 58E. The proximal end portion 34E of the inner elongate body 30E includes at least one protrusion 60E. The protrusion 60E is configured to be selectively received within at least one of the multiple apertures 58E to adjustably couple the outer elongate body 22E to the inner elongate body 30E. The protrusions 60E may be a known spring-loaded protrusion or other suitable construction. In this embodiment, the inner elongate body 30E can be coupled to the outer elongate body 22E at a selected aperture 58E to lock the medical device 20E in an extended configuration as shown in FIG. 9. In the extended configuration, a flange 54E on the proximal end portion 34E of the inner elongate body 30E contacts a flange 52E on the distal end portion 24E of the outer elongate body 22E.

To unlock the medical device 20E, the protrusions 60E are moved out of the apertures 58E and into clearance areas 82E (see FIG. 10) within the outer elongate body 22E. This allows the outer elongate body 22E to be collapsed over the inner elongate body 30E to adjust the length of the medical device 20E. A handle 28E on the outer elongate body 22E can then be positioned at a selected height above the body surface of the patient.

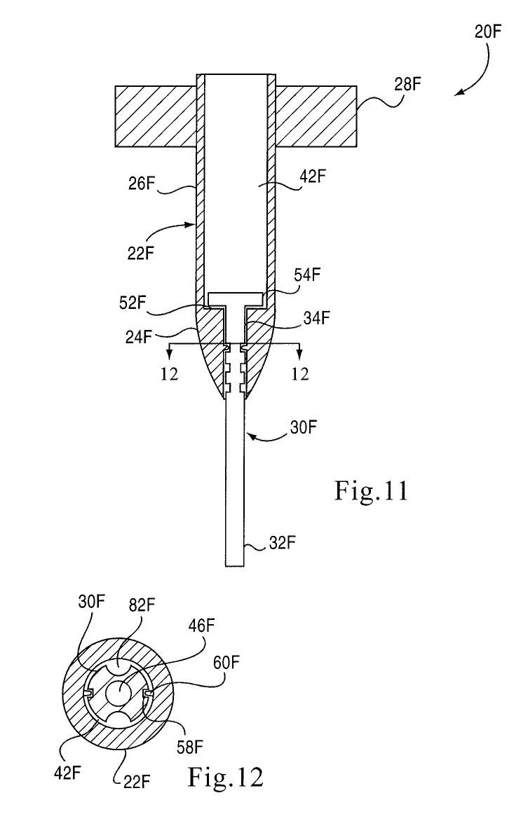

FIGS. 11 and 12 illustrate an embodiment of a medical device 20F that is similar to the medical device 20E. In this embodiment, at least one protrusion 60F is disposed on an inner wall of distal end portion 24F of an outer elongate body 22F, and multiple apertures 58F are defined on a proximal end portion 34F of an inner elongate body 30F. As with the previous embodiment, the at least one protrusion 60F is configured to be selectively received within at least one of the multiple apertures 58F to adjustably couple the outer elongate body 22F to the inner elongate body 30F. The protrusions 60F may be a spring-loaded protrusion or other suitable construction. In this embodiment, the inner elongate body 30F can be coupled to the outer elongate body 22F at a selected aperture 58F to lock the medical device 20F in an extended configuration as shown in FIG. 11. In the extended configuration, a flange 54F on the proximal end portion 34F of the inner elongate body 30F contacts a flange 52F on the distal end portion 24F of the outer elongate body 22F.

To unlock the medical device 20F, the protrusions 60F are moved out of the apertures 58F and into clearance areas 82F (see FIG. 12) within the inner elongate body 30F. This allows the outer elongate body 22F to be collapsed over the inner elongate body 30F to adjust the length of the medical device 20F. A handle 28F on the outer elongate body 22F can then be positioned at a selected height above the body surface of the patient.

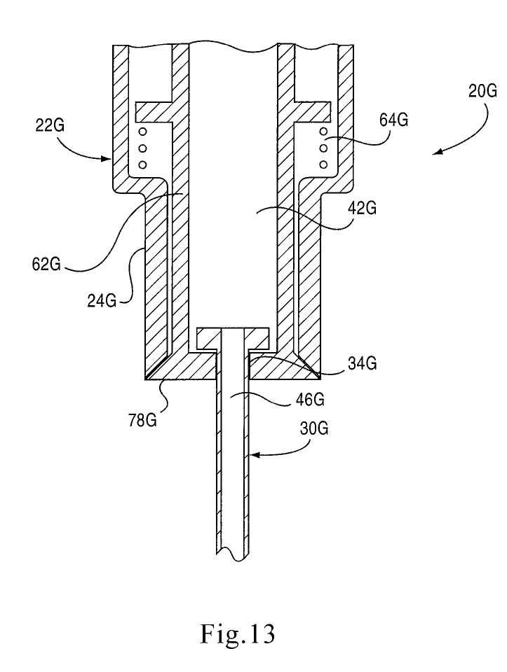

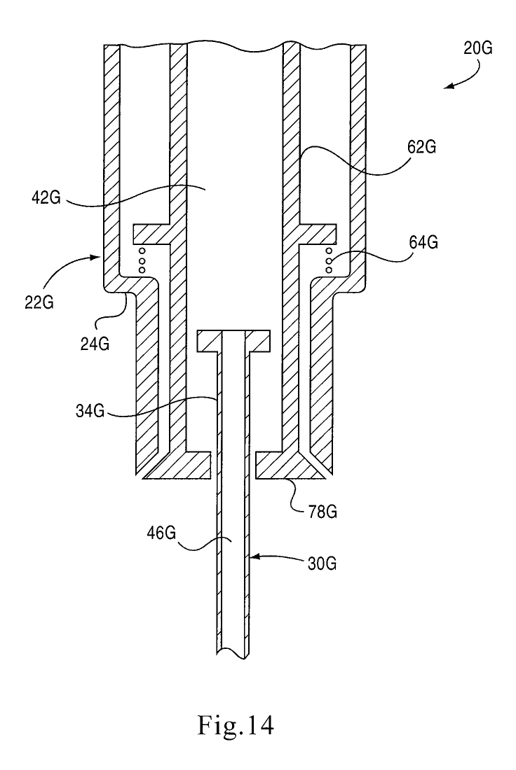

FIG. 13 illustrates a cross-sectional view of a portion of another embodiment of a medical device 20G in a closed configuration, and FIG. 14 illustrates the medical device 20G in an open configuration. Medical device 20G includes an outer elongate body 22G and an inner elongate body 30G. The outer elongate body includes a proximal end portion (not shown) and a distal end portion 24G, and defines a lumen 42G. The inner elongate body 30G includes a proximal end portion 34G and a distal end portion (not shown), and defines a lumen 46G. The medical device 20G is similar to the previous embodiments except this embodiment includes a collar 62G and a spring element 64G. The collar 62G is coupled to the distal end portion 24G of the outer elongate body 22G. The collar 62G is configured to couple the distal end portion 24G of the outer elongate body 22G to the proximal end portion 34G of the inner elongate body 30G. The spring element 64G biases the collar 62G into a closed configuration in which a distal end portion 78G of the collar 62G is in contact with the inner elongate body 30G as shown in FIG. 13. The collar 62G holds the inner elongate body 30G at a selected axial or longitudinal position relative to the outer elongate body 22G. When the collar 62G is pushed distally, the distal end portion 78G of the collar 62G spreads apart to an open configuration, as shown in FIG. 14. In the open configuration, the outer elongate body 22G is able to slidably (e.g., telescopically) move over inner elongate body 30G to adjust the length of the medical device 20G. Thus, in the open configuration, a handle (not shown in FIGS. 13 and 14) on the outer elongate body 22G can be positioned at a selected location above a body surface of a patient. At the selected location, the collar 62G is released, which again biases the distal end portion 78G of the collar 62G into contact with the inner elongate body 30G.

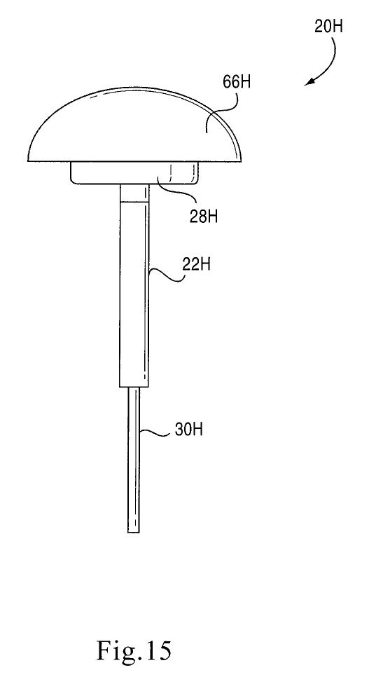

FIG. 15 illustrates a medical device 20H including an outer elongate body 22H and an inner elongate body 30H. The medical device 20H performs substantially the same functions as the previous embodiments. Medical device 20H illustrates an embodiment where the inner elongate body 30H includes a handle 66H that releasably and matingly couples to a handle 28H on the outer elongate body 22H. In this embodiment, a tool, such as a stylet or other device can be used to initially puncture the tissue and create an access opening. The medical device 20H can then be inserted into the tissue through the access opening.

The medical device for any of the embodiments may be constructed with any suitable material used for such a medical device. For example, the outer elongate body may be constructed with stainless steel or alternatively with a suitable plastic material, as it does not have to penetrate bone. The inner elongate body and the tool may each be constructed with stainless steel or other suitable material that is sufficiently strong to allow penetration of hard tissue (e.g., bone)e. Alternatively, the inner elongate body and the tool may each be constructed with a suitable plastic material where the medical device is used to penetrate soft tissue.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. Thus, the breadth and scope of the invention should not be limited by any of the above-described embodiments, but should be defined only in accordance with the following claims and their equivalents. While the invention has been particularly shown and described with reference to specific embodiments thereof, it will be understood that various changes in form and details may be made.

For example, some embodiments may include a tool having a sharp tip to puncture the tissue. In other embodiments, the inner elongate body will include a sharp tip or edge to puncture the tissue. In which case, a tool, such as a stylet, may or may not be included. In addition, in some embodiments, neither the tool nor the inner elongate body will have a sharp tip. In such an embodiment, another medical device can be used to puncture the tissue prior to inserting a medical device described herein. Any of the embodiments described herein can include any combination of the various components.

Also, any of the coupling methods described herein may be used, as well as other known coupling methods, to releasably couple the outer elongate body to the inner elongate body. In some embodiments, the inner elongate body and the outer elongate body may include a coupling that locks them into the extended configuration. In other embodiments, the inner elongate body and the outer elongate body may only be coupled such that rotation relative to each other is prevented, and a tool is used to secure the medical device in the extended configuration. In addition, the sharp tip or edge, whether on the tool or the inner elongate body, may be a variety of different shapes and sizes.

In use, the medical device may be adjusted such that the handle on the outer elongate body is positioned at a selected height above the body surface of the patient. In some embodiments, the handle may be positioned flush with the body surface of the patient. The medical device can be used as an access cannula for inserting a variety of different medical devices or tools to perform a medical procedure.

What is claimed is:

1. An apparatus, comprising:
an outer elongate body including a lumen extending from a proximal portion to a distal portion of the outer elongate body; and
an inner elongate body slidably received within the lumen of the outer elongate body, a distal portion of the inner elongate body is configured to be inserted into a tissue, the outer elongate body and the inner elongate body collectively defining a channel to removably receive a tool when the outer elongate body and the inner elongate body are slidably extended from each other, at least one of the tool and the inner elongate body being configured to puncture the tissue.

2. The apparatus of claim 1, wherein the tissue is a vertebra.

3. The apparatus of claim 1, wherein the tissue includes at least an outer soft tissue layer and an inner tissue layer.

4. The apparatus of claim 1, wherein the inner elongate body is configured to at least partially penetrate an inner tissue layer when slidably extended from the outer elongate body and inserted into the tissue, and the outer elongate body is configured to be slid over at least a portion of the inner elongate body as a portion of the outer elongate body is advanced into an outer soft tissue layer after the inner elongate body has penetrated into the inner tissue layer.

5. The apparatus of claim 1, wherein the inner elongate body is configured to at least partially penetrate an inner tissue layer when slidably extended from the outer elongate body and inserted into the tissue, the inner tissue layer includes at least one of a boney tissue or a collagen based tissue.

6. The apparatus of claim 1, wherein the inner elongate body is threadedly coupled to the outer elongate body.

7. The apparatus of claim 1, wherein the distal end portion of the outer elongate body includes a tapered portion configured to matingly receive a proximal end portion of the inner elongate body.

8. The apparatus of claim 1, wherein the distal end portion of the outer elongate body defines a plurality of apertures, a proximal end portion of the inner elongate body includes at least one protrusion configured to be selectively received within at least one of the plurality of apertures to adjustably couple the outer elongate body to the inner elongate body.

9. The apparatus of claim 1, further comprising:
a collar coupled to the distal end portion of the outer elongate body, the collar configured to adjustably couple the outer elongate body to the inner elongate body.

10. The apparatus of claim 1, further comprising:
a collar and a spring coupled to the collar, the collar coupled to the distal end portion of the outer elongate body and configured to adjustably couple the outer elongate body to the inner elongate body, the spring configured to bias the collar into contact with the inner elongate body.

11. The apparatus of claim 1, wherein the inner elongate body defines a lumen extending from a proximal end portion of the inner elongate body to the distal end portion of the inner elongate body, the apparatus further comprising:
the tool, the tool having a sharp tip configured to puncture the tissue, the tool extending through the lumen of the inner elongate body and the lumen of the outer elongate body.

12. The apparatus of claim 1, wherein the inner elongate body defines a lumen extending from a proximal end portion of the inner elongate body to the distal end portion of the inner elongate body, the inner elongate body further defines an opening on the distal end portion, the opening in communication with the lumen, the inner elongate body including a sharp edge on the distal end portion, the sharp edge on the distal end portion of the inner elongate body configured to puncture a tissue, the apparatus further comprising:
the tool, the tool extending through the lumen of the outer elongate body and the lumen of the inner elongate body to the opening defined by the inner elongate body.

13. The apparatus of claim 1, wherein the distal end portion of the outer elongate body defines a flange on an inner wall, a proximal end portion of the inner elongate body defines a flange on an outer wall, the flange of the proximal end portion of the inner elongate body configured to contact the flange of the distal end portion of the outer elongate body.

14. An apparatus, comprising:
an outer elongate body including a proximal end portion and a distal end portion; and
an inner elongate body including a proximal end portion and a distal end portion, the inner elongate body being configured to be slidably received within the outer elongate body, the distal end portion of the inner elongate body configured to be inserted into a tissue, the inner elongate body being configured to couple to the outer elongate body such that the outer elongate body and the inner elongate body are collectively rotatable without the outer elongate body and the inner elongate body rotating with respect to each other while in an extended configuration.

15. The apparatus of claim 14, wherein the distal end portion of the outer elongate body is configured to be threadedly coupled to the inner elongate body.

16. The apparatus of claim 14, wherein the distal end portion of the outer elongate body includes a tapered portion, the tapered portion of the outer elongate body configured to complimentarily fit to a tapered portion of the inner elongate body.

17. The apparatus of claim 14, wherein the distal end portion of the outer elongate body defines a plurality of apertures, the proximal end portion of the inner elongate body includes at least one protrusion to be selectively received within at least one of the plurality of apertures to adjustably couple the outer elongate body to the inner elongate body.

18. The apparatus of claim 14, wherein the tissue is a vertebra.

19. An apparatus, comprising:
an outer elongate body including a proximal end portion and a distal end portion;
an inner elongate body including a proximal end portion and a distal end portion, the inner elongate body being configured to be slidably received within the outer elongate body, the distal end portion of the inner elongate body configured to be inserted into a tissue, the outer elongate body and the inner elongate body collectively defining a channel having a length while the outer elongate body and the inner elongate body are in a slidably extended configuration; and
a tool configured to be slidably received within the outer elongate body and the inner elongate body, the tool having a proximal end and a distal end, the distal end of the tool including a sharp tip configured to puncture a tissue, the tool having a length greater than the length of the channel while the outer elongate body and the inner elongate body are in the slidably extended configuration.

20. The apparatus of claim 19, wherein the proximal end portion of the inner elongate body is threadedly coupled to the distal end portion of the outer elongate body.

21. The apparatus of claim 19, wherein the distal end portion of the outer elongate body includes a tapered portion configured to matingly receive the proximal end portion of the inner elongate body.

22. The apparatus of claim 19, wherein the distal end portion of the outer elongate body defines a plurality of apertures, each aperture from the plurality of apertures being positioned on an inner wall of the distal end portion of the outer elongate body, the inner elongate body includes at least one protrusion configured to be selectively received within at least one of the plurality of apertures to adjustably couple the outer elongate body to the inner elongate body.

23. The apparatus of claim 19, wherein the distal end portion of the outer elongate body defines a flange on an inner wall, the proximal end portion of the inner elongate body defines a flange on an outer wall, the flange defined by the proximal end portion of the inner elongate body configured to contact the flange defined by the distal end portion of the outer elongate body.

24. The apparatus of claim 19, further comprising:
a collar coupled to the distal end portion of the outer elongate body, the collar configured to adjustably couple the outer elongate body to the inner elongate body.

25. The apparatus of claim 19, further comprising:
a collar and a spring coupled to the collar, the collar coupled to the distal end portion of the outer elongate body and configured to adjustably couple the outer elongate body to the inner elongate body, the spring configured to bias the collar into contact with the inner elongate body.

26. The apparatus of claim 19, further comprising:
a locking mechanism for securing the position of the inner elongate body in relation to the outer elongate body.

27. The apparatus according of claim 19, wherein the tool includes a locking mechanism for securing the position of the inner elongate body in relation to the outer elongate body.

28. The apparatus of claim 19, wherein the tool is a stylet.

29. The apparatus of claim 19, wherein the tissue is a vertebral body.

30. A method of positioning a cannula to perform a medical procedure, comprising:
inserting a cannula assembly into a tissue such that a distal end of an inner elongate body of the cannula assembly is positioned in a portion of the tissue; and
slidably collapsing an outer elongate body of the cannula assembly over the inner elongate body of the cannula assembly such that a handle on a distal end of the outer elongate body is positioned at a selected height outside of a body of a patient.

30. The method of claim 30, further comprising:
removing a tool from the cannula assembly after the inserting and the collapsing.

31. The method of claim 30, further comprising:
removing a tool from the cannula assembly.

32. The method according to claim 30 further comprising:
introducing a medical device into the tissue through the inner elongate body.

33. The method of claim 30, wherein the collapsing includes turning the handle on the outer elongate body to move the outer elongate body over the inner elongate body.

34. The method of claim 30, further comprising:
coupling the outer elongate body to the inner elongate body such that the outer elongate body and the inner elongate body are collectively rotatable without the outer elongate body and the inner elongate body rotating with respect to each other.

35. An apparatus comprising:
an inner elongate body slidably disposed within a lumen of an outer elongate body, the inner elongate body includes a lumen, and at least a distal portion of the inner elongate body is configured to penetrate a tissue, the inner elongate body configured to receive a tool within the lumen of the inner elongate body; and
a locking mechanism configured to allow a user to electively secure the inner elongate body in relation to the outer elongated body.

36. The apparatus of claim 35, wherein the tissue includes an inner tissue layer and an outer soft tissue layer, the inner elongate body is configured to at least partially penetrate the inner tissue layer, the outer elongate body is configured to be slid over at least portion of the inner elongate body as a portion of the outer elongate body is advanced into the outer soft tissue layer after the inner elongate body is deployed into the inner tissue layer.

37. The apparatus of claim 35, wherein the tissue includes an inner tissue layer and an outer soft tissue layer, the inner tissue layer includes at least one of a boney tissue or a collagen based tissue.

38. The apparatus of claim 35, further comprising:
a tool configured for placement within the lumen of the inner elongate body, a distal end of the tool is configured to penetrate the tissue.

39. The apparatus of claim 35, further comprising:
a tool configured to be disposed within the lumen of the inner elongate body, the tool configured to be coupled to the outer elongate body to secure the outer elongate body and the inner elongate body in an extended configuration.

40. The apparatus of claim 35, further comprising:
a tool configured for placement within the lumen of the inner elongate body, a distal end of the tool being configured to extend through the lumen of the inner elongate body and exit at a distal end of the inner elongate body and puncture the tissue.

41. The apparatus of claim 40, wherein the inner elongate body is configured to penetrate a boney tissue.
